(19) United States
(12) Patent Application Publication
(10) Pub. No.: US 2021/0016113 A1
(43) Pub. Date: Jan. 21, 2021

(54) CLOSED LOOP NEUROSTIMULATION OF LARGE-SCALE BRAIN NETWORKS

(71) Applicant: X Development LLC, Mountain View, CA (US)

(72) Inventors: Thomas Peter Hunt, Oakland, CA (US); Matthew Dixon Eisaman, Port Jefferson, NY (US); Sarah Ann Laszlo, Mountain View, CA (US)

(21) Appl. No.: 16/931,778

(22) Filed: Jul. 17, 2020

Related U.S. Application Data
(60) Provisional application No. 62/875,171, filed on Jul. 17, 2019.

(51) Int. Cl. A61N 7/00; A61B 8/00

(52) U.S. Cl. CPC A61N 7/00; A61N 2007/0078; A61N 2007/0026; A61B 8/461

(57) ABSTRACT

Closed-loop neurostimulation of large-scale brain networks includes a neurostimulation headset having at least two ultrasound transducer modules configured to generate within a first time period, a first focused ultrasound wave at a region within a portion of a subject's brain, one or more sensors configured to measure, within the first time period, a response from the portion of the subject's brain in response to the first focused ultrasound wave, and an electronic controller in communication with the at least two emitters and the one or more sensors configured to dynamically adjust, based on the measured response from the portion of the subject's brain, a power level of one or more of the at least two ultrasound transducer modules to generate a second focused ultrasound wave at the region within the portion of the subject's brain.

### CROSS-REFERENCE TO RELATED APPLICATION

This application claims the benefit of U.S. Application No. 62/875,171, filed Jul. 17, 2019, the contents of which are incorporated by reference herein.

### FIELD

This specification relates to closed loop neurostimulation of large-scale brain networks.

### BACKGROUND

Stimulation of the brain in humans is typically performed using an “open loop,” where control of the stimulation is independent of the results of the stimulation. In general, neurostimulation techniques performed on the brain are performed without feedback. Stimulation is also performed with respect to a generic position relative to a subject's head, and typically is not based on the particular subject's brain morphology or measurements of the particular subject's brain activity.

### SUMMARY

Brain stimulation is now thought to treat conditions such as depression, anxiety, and obsessive-compulsive disorder, and there is growing evidence that stimulation can improve memory or modulate attention and mindfulness. Stimulation is typically performed with electric or magnetic fields, or ultrasound, in an “open loop,” i.e., without feedback. Treatments are generally performed at a generic position relative to a user's head, instead of being based on individual brain morphology or measurements of an individual's brain, and are not tailored to the needs of each particular user at each particular moment of use.

The proposed methods perform closed loop stimulation of the brain and can target large-scale brain networks in real-time, while contemporaneously or near-contemporaneously collecting measurements and adjusting the stimulation based on the measurements as they are taken. The stimulation can be done through focused ultrasound waves, multiple-source direct current, alternating current, and/or interfering kHz-frequency electric fields, e.g., on the order of Volts. The stimulation emitted or generated can be complex fields that use interference or focusing effects to structure fields from multiple emitters or sources. The emitters or sources can be, for example, transducers for focused ultrasound stimulation or electrodes for electrical stimulation. The response to the stimulation that is measured can be, for example, in the frequency range of Hz to many tens of Hz (e.g., 15 Hz) and on the order of μV. The brain's response is measured and used as feedback to dynamically adjust the applied stimulation, creating a continuous, closed loop system that is customized for each user. The closed loop system also enables phase locking between large-scale brain networks to be measured and stimulation to be applied with a known phase delay, for example, in-phase with contemporaneous or near-contemporaneous brain signal measurements.

In addition to sensing responses to the stimulation, the methods can include measuring brain activity and function through optical, electrical (e.g., electroencephalogram (EEG)), ultrasonic, and/or magnetic (e.g., magnetoencephalogram (MEG)) techniques, and can include measuring other vital signs of a user, including heart rate, respiratory rate, temperature, blood pressure, etc. In addition to measuring brain activity, brain anatomy can be measured in high resolution with magnetic resonance imaging (MRI), in order to reduce the solution space for approximating the EEG inverse problem, where there can be multiple patterns of stimulation that could have caused a particular measured response via EEG.

Machine learning models can be used to analyze the measured response. In some cases, the models can be applied to the proposed method to map out brain connectivity, conductivity, and functionality. The models can be used for tomography of the brain, producing electrical conductivity volume maps based on electrical measurements of known input fields. Forward modelling can be accomplished on the basis of anatomical MRI and/or computed tomography as just described. Inverse modelling can also be conducted by using measured responses to approximate brain networks that could produce the measured responses. The methods can use additional biomarker inputs to determine the stimulus or feedback. For example, the methods can use vital signs of the user as additional input to the model to improve the accuracy of the model and to personalize the models to the user.

The proposed methods can be implemented in the form of a headset with multiple emitters that attach to a user's head. The headset may be used under the supervision of a medical health professional. The headset includes safety features that allow the headset to be used without the supervision of a medical health professional. For example, the range of frequencies and intensities of the electrical, ultrasonic, and/or magnetic stimulation applied through the emitters can be restricted to prevent delivering seizure inducing stimulus. The headset measurements may confirm the user's unique biometric signature to limit dosing. The headset can constantly monitor for pre-epileptic signatures, and cut stimulation or apply anti-epileptic stimulation. The headset can be used in non-clinical situations to aid in relaxation and meditation, to stimulate creativity, and to increase focus. In some implementations, the headset can be used for clinical purposes to treat neural conditions.

In one general implementation, the proposed method includes generating, within a first time period and by at least two emitters placed on a subject's head, an electric field comprising an interfering region within a portion of the subject's brain, the interfering region having a beat frequency less than 100 Hz, measuring, within the first time period and by one or more sensors, a response from the portion of the subject's brain in response to the interfering region of the electric field, dynamically adjusting, based on the measured response from the portion of the subject's brain, the electric field.

In another general implementation, the proposed method includes generating, within a first time period and by at least two emitters placed on a subject's head, an electric field within a portion of the subject's brain having a frequency less than 100 Hz, measuring, within the first time period and by one or more sensors, a response from the portion of the subject's brain in response to the electric field, and dynamically adjusting, based on the measured response from the portion of the subject's brain, the electric field.

In another general implementation, the proposed method includes generating, within a first time period and by at least two emitters placed on a subject's head, an electric field within a portion of the subject's brain having a frequency less than 100 Hz, measuring, within a second time period that is a threshold amount of time from the first period, and by one or more sensors, a response from the portion of the subject's brain in response to the electric field, calculating, based on the measured response from the portion of the subject's brain in response to the electric field, an adjusted electric field, and dynamically adjusting, based on the measured response from the portion of the subject's brain, the electric field to generate the adjusted electric field.

In another general implementation, the proposed method includes imaging, by at least two emitters placed on a subject's head, the subject's brain, generating, based on the imaging, within a first time period, and by at least two emitters placed on a subject's head, an electric field comprising an interfering region within a portion of the subject's brain, the interfering region having a beat frequency less than 100 Hz, measuring, within the first time period and by one or more sensors, a response from the portion of the subject's brain in response to the interfering region of the electric field, determining, by analyzing the measured response using a machine learning model, an adjusted electric field, and dynamically adjusting, based on the measured response from the portion of the subject's brain, the electric field to generate the adjusted electric field.

In another implementation, a neurostimulation headset includes at least two ultrasound transducer modules configured to generate, within a first time period, a first focused ultrasound wave at a region within a portion of a subject's brain, one or more sensors configured to measure, within the first time period, a response from the portion of the subject's brain in response to the first focused ultrasound wave, and an electronic controller in communication with the at least two emitters and the one or more sensors configured to dynamically adjust, based on the measured response from the portion of the subject's brain, a power of one or more of the at least two ultrasound transducer modules to generate a second focused ultrasound wave at the region within the portion of the subject's brain.

In some implementations, sensors include an imaging array that generates imaging ultrasound waves at a first power level sufficient to produce an imaging effect of the region within the portion of the subject's brain. In some implementations, the second focused ultrasound wave is at a second power level sufficient to provide a therapeutic effect for the subject. In some implementations, the electronic controller is further configured to calculate, based on the measured response from the portion of the subject's brain in response to the first focused ultrasound wave, an adjusted focused ultrasound wave and dynamically adjust, based on the measured response from the portion of the subject's brain, the focused ultrasound wave to generate the second focused ultrasound wave. In some implementations, dynamically adjusting the focused ultrasound wave to generate the second focused ultrasound wave includes determining, based on the location of the region and a machine learning model, at least one focusing parameter for the one or more ultrasound transducer modules.

Systems for implementing the proposed method can be embodied in various form factors. In one general implementation, the system can be embodied in a neurostimulation headset that includes at least two emitters configured to generate, within a first time period, an electric field comprising an interfering region within a portion of the subject's brain, the interfering region having a beat frequency less than 100 Hz, one or more sensors configured to measure, within the first time period, a response from the portion of the subject's brain in response to the interfering region of the electric field, and an electronic controller in communication with the at least two emitters and the one or more sensors configured to dynamically adjust, based on the measured response from the portion of the subject's brain, the electric field.

In another general implementation, the system can be embodied in a neurostimulation headset that includes at least two emitters configured to generate, within a first time period, an electric field within a portion of the subject's brain having a frequency less than 100 Hz, one or more sensors configured to measure, within the first time period, a response from the portion of the subject's brain in response to the electric field, and an electronic controller in communication with the at least two emitters and the one or more sensors configured to dynamically adjust, based on the measured response from the portion of the subject's brain, the electric field.

The details of one or more implementations are set forth in the accompanying drawings and the description, below. Other potential features and advantages of the disclosure will be apparent from the description and drawings, and from the claims.

### BRIEF DESCRIPTION OF THE DRAWINGS

FIG. 1 is a diagram of an example configuration of a closed loop neurostimulation system.

FIG. 2 is a diagram of an example machine learning process for closed loop neurostimulation of large-scale brain networks.

FIG. 3 is a flow chart of an example process of closed loop neurostimulation of large-scale brain networks.

Like reference numbers and designations in the various drawings indicate like elements. The components shown here, their connections and relationships, and their functions, are meant to be examples only, and are not meant to limit the implementations described and/or claimed in this document.

### DETAILED DESCRIPTION

Stimulation of large-scale brain networks—various sets of synchronized brain areas linked together by brain function—is now thought to treat neurological disorders, such as anxiety disorders, trauma and stressor-related disorders, panic disorders, and mood disorders. Additionally, there has been growing evidence of the effects of neurostimulation of large-scale brain networks on a subject's memory or attention. In general, conventional neurostimulation of brain networks is not automatically tailored for particular subjects and their needs, and does not take into account brain activity that occurs in response to the stimulation. These methods typically only perform stimulation at a generic position with respect to a subject's head, and are not based on a particular subject's brain morphology or activity.

The proposed methods and systems perform closed loop stimulation of the brain and allows for stimulation of large-scale brain networks in real-time, while contemporaneously or near-contemporaneously collecting measurements and adjusting the stimulation based on the measurements as they are taken. The brain activity and function measurements can be used with statistical and/or machine learning models to analyze the response of the subject's brain to the stimulation. In some implementations, the measurements can be used to map out brain electrical conductivity, connectivity, and functionality to personalize stimulation to a particular subject.

For example, the proposed methods can include continuously providing stimulation to a particular area of a subject's brain, contemporaneously or near-contemporaneously recording brain activity detected by sensors, reconstructing the location, amplitude, frequency, and phase of large-scale brain activity in response to the stimulation, designing stimulation field patterns to modify the reconstructed brain activity, and applying the designed stimulation field patterns.

The proposed methods and systems can be implemented automatically. For example, the controller can automatically determine the connectivity or resting state activity of a particular subject's brain to tailor stimulation patterns and detection techniques to the particular subject's brain.

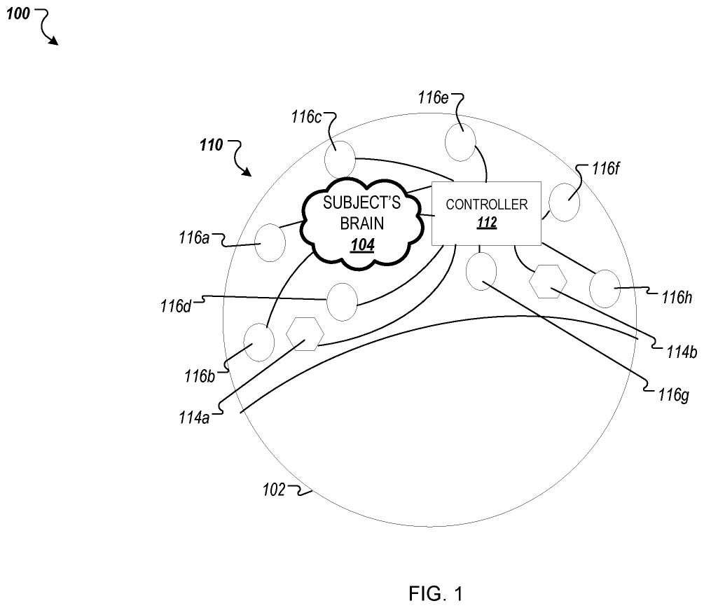

FIG. 1 is a diagram of an example configuration 100 of a closed loop neurostimulation system 110. System 110 provides neurostimulation of large-scale brain networks. For example, system 110 can be used to stimulate a target area of a subject's brain and, based on read out data of measured brain activity in response to the stimulation, the system 110 can adjust various parameters of the stimulation of the target area.

In this particular example, system 110 is in the form of a wearable headpiece that can be placed on a subject's head. In some implementations, system 110 can be in the form of a network of individual emitters and sensors that can be placed on the subject's head or a system that holds individual emitters and sensors in fixed positions around the subject's head. For example, the emitters can be electrodes. The electrodes may be wet or dry.

In this particular example, system 110 can be used without an external power source. For example, system 110 can include an internal power source. The internal power source can be rechargeable and/or replaceable. For example, system 110 can include a replaceable, rechargeable battery pack that provides power to the emitters and sensors.

Subject 102 is a human subject of neurostimulation.

A focal spot, or target area, within subject's brain 104 can be targeted. The target area can be, for example, a specific large-scale brain network associated with a particular state of subject's brain 104. In some implementations, the target area can be automatically selected based on detection data. For example, the system 110 can adjust the targeted area within subject's brain 104 based on detected brain activity. In some implementations, the target area can be selected manually based on a target reaction from subject's brain 104 or a target reaction from other body parts of the subject.

Neurostimulation system 110 is shown to include a controller 112, sensors 114a, 114b, and 114c (collectively referred to as sensors 114), and emitters 116a, 116b, 116c, 116d, 116e, 116f, 116g, and 116h (collectively referred to as emitters 116). System 110 is configured to provide closed loop neurostimulation of large-scale brain networks through simultaneous use of multiple emitters.

System 110 allows contemporaneous or near-contemporaneous detection and stimulation, facilitating a closed loop system that is able to target large-scale brain networks of subject's brain 104 in real time and make adjustments to the stimulation based on the detected data. Detection and stimulation may alternate with a period of seconds or less to enable the closed loop system. Detection and stimulation signals can be multiplexed. The closed loop system also allows system 110 to enable phase locking between large-scale brain networks to be measured, such that system 110 can apply stimulation to a target area of subject's brain 104 with a known phase delay from a reference signal. For example, controller 112 can apply stimulation, through electrical fields, to a target area of subject's brain 104 in-phase with contemporaneous or near-contemporaneous brain signal measurements.

Sensors 114 detect activity of subject's brain 104. Detection can be done using electrical, optical, and/or magnetic techniques, such as EEG, MEG, and MRI, among other types of detection techniques. For example, sensors 114 can include non-invasive sensors such as EEG sensors, MEG sensors, among other types of sensors. In this particular implementation, sensors 114 are EEG sensors. Sensors 114 can include temperature sensors, infrared sensors, light sensors, heart rate sensors, and blood pressure monitors, among other types of sensors. In addition to detecting activity of the subject's brain 104, sensors 114 can collect and/or record the activity data and provide the activity data to controller 112.

Sensors 114 can, for example, include an imaging array that generates ultrasound waves at a power level sufficient for producing an imaging effect of a target region within a portion of subject's brain 104.

Sensors 114 can perform optical detection such that detection does not interfere with the frequencies generated by emitters 116. For example, sensors 114 can perform near-infrared spectroscopy (NIRS) or ballistic optical imaging through techniques such as coherence gated imaging, collimation, wavefront propagation, and polarization to determine time of flight of particular photons. Additionally, sensors 114 can collect biometric data associated with subject 102. For example, sensors 114 can detect the heart rate, eye movement, and respiratory rate, among other biometric data of the subject 102.

Sensors 114 provide the collected brain activity data and other data associated with subject 102 to controller 112.

Emitters 116 generate one or more electric fields at a target area within a subject's brain 104. System 110 includes multiple emitters 116, which can generate multiple fields that create an interfering region at a focal point, such as a target area within subject's brain 104. Emitters 116 can be, for example, electrodes. Emitters 116 can be powered by direct current or alternating current. Emitters 116 can be identical to each other. In some implementations, emitters 116 can include emitters made of different materials.

In some implementations, sensors 114 can include emitters that emit and detect electrical activity within the subject's brain 104. For example, emitters 116 can include one or more of sensors 114. In some implementations, emitters 116 include each of sensors 114; the same set of emitters can perform the stimulation and detection of brain activity in response to the stimulation. In some implementations, one subset of emitters may be dedicated to stimulation and another subset dedicated to detection. In some implementations, the stimulation system, i.e., emitters 116, and the detection system, i.e., sensors 114, are electromagnetically or physically shielded and/or separated from each other such that fields from one system do not interfere with fields from the other system. In some implementations, system 110 allows for contemporaneous or near-contemporaneous stimulation and measurement through, for example, the use of high performance filters that allow for high frequency stimulation at a high amplitude during low noise detection.

Controller 112 includes one or more computer processors that control the operation of various components of system 110, including sensors 114 and emitters 116 and components external to system 110, including systems that are integrated with system 110.

Controller 112 generates control signals for the system 110 locally. The one or more computer processors of controller 112 continually and automatically determine control signals for the system 110 without communicating with a remote processing system. For example, controller 112 can receive brain activity feedback data from sensors 114 in response to stimulation from emitters 116 and process the data to determine control signals and generate control signals for emitters 116 to alter or maintain one or more fields generated by emitters 116 within the target area of subject's brain 104.

Controller 112 controls sensors 114 to collect and/or record data associated with subject's brain 104. For example, sensors 114 can collect and/or record data associated with stimulation of subject's brain 104. In some implementations, controller 112 can control sensors 114 to detect the response of subject's brain 104 to stimulation generated by emitters 116. Sensors 114 can also measure brain activity and function through optical, electrical, and magnetic techniques, among other detection techniques.

Controller 112 is communicatively connected to sensors 114. In some implementations, controller 112 is connected to sensors 114 through communications buses with sealed conduits that protect against solid particles and liquid ingress. In some implementations, controller 112 transmits control signals to components of system 110 wirelessly through various wireless communications methods, such as RF, sonic transmission, electromagnetic induction, etc.

Controller 112 can receive feedback from sensors 114. Controller 112 can use the feedback from sensors 114 to adjust subsequent control signals to system 110. The feedback, or subject's brain 104's response to stimulation generated by emitters 116 can have frequencies on the order of tens of Hz and voltages on the order of μV. Subject's brain 104's response to stimulation generated by emitters 116 can be used to dynamically adjust the stimulation, creating a continuous, closed loop system that is customized for subject 102.

Controller 112 can be communicatively connected to sensors other than sensors 114, such as sensors external to the system 110, and uses the data collected by sensors external to the system 110 in addition to the sensors 114 to generate control signals for the system 110. For example, controller 112 can be communicatively connected to biometric sensors, such as heart rate sensors or eye movement sensors, that are external to the system 110.

Controller 112 can accept input other than EEG data from the sensors 114. The input can include sensor data from sensors separate from system 110, such as temperature sensors, light sensors, heart rate sensors, and blood pressure monitors, among other types of sensors. In some implementations, the input can include user input. In some implementations, and subject to safety restrictions, a subject can adjust the operation of the system 110 based on the user's comfort level. For example, the subject can provide direct input to the controller 112 through a user interface. In some implementations, controller 112 receives sensor information regarding the condition of a user. For example, sensors monitoring the heart rate, respiratory rate, temperature, blood pressure, etc., of a subject can provide this information to controller 112. Controller 112 can use this sensor data to automatically control system 110 to alter or maintain one or more fields generated within the target area of subject's brain 104.

Controller 112 uses data collected by sensors 114 and sources separate from system 110 to reconstruct characteristics of brain activity detected in response to stimulation from emitters 116, including the location, amplitude, frequency, and phase of large-scale brain activity. For example, controller 112 can use individual MRI brain structure maps to calculate electric field locations within a particular brain, such as subject's brain 104.

Controller 112 controls the selection of which of emitters 116 to activate for a particular stimulation pattern. Controller 112 controls the voltage, frequency, and phase of electric fields generated by emitters 116 to produce a particular stimulation pattern. In some implementations, controller 112 uses time multiplexing to create various stimulation patterns of electric fields using emitters 116. In some implementations, controller 112 turns on various combinations of emitters 116, which may have differing operational parameters (e.g., voltage, frequency, phase) to create various stimulation patterns of electric fields.

Controller 112 selects which of emitters 116 to activate and controls emitters 116 to generate fields in a target area of subject's brain 104 based on detection data from sensors 114 and stimulation parameters for subject 102. In some implementations, controller 112 selects particular emitters based on the position of the target area. For example, controller 112 can select opposing emitters closest to the target area within subject's brain 104. In some implementations, controller 112 selects particular emitters based on the stimulation to be applied to the target area. For example, controller 112 can select emitters capable of producing a particular voltage or frequency of electric field at the target area.

Controller 112 operates multiple emitters 116 to generate electric fields at the target area of subject's brain 104. Controller 112 operates multiple emitters 116 to generate electric fields using direct current or alternating current. Controller 112 can operate multiple emitters 116 to create interfering electric fields that interfere to produce fields of differing frequencies and voltage. For example, controller 112 can operate two opposing emitters 116 (e.g., emitters 116a and 116h) to generate two electric fields having frequencies on the order of kHz that interfere to produce an interfering electric field having a frequency on the order of Hz. Controller 112 can control operational parameters of emitters 116 to generate ultrasonic waves, magnetic fields, or electric fields. For example, controller 112 can control emitters 116 to generate electric fields that interfere to create an interfering field having a particular beat frequency. In some implementations, the beat frequency of the interfering field can be less than 100 Hz. The voltages of the electric fields generated by emitters 116 are on the order of 0.5 to a few Volts.

In some implementations, the controller 112 operates focusing elements such for focusing the ultrasonic waves generated by emitters 116. For example, controller 112 can operate focusing elements such as axicons or Fresnel zone plates integrated with the transducers.

Controller 112 can achieve directional signal transmission or reception through beamforming by combining elements in an antenna array such that signals at particular angles experience constructive interference while others experience destructive interference in order to achieve spatial selectivity. For example, based on the ultrasound imaging or measurements, controller 112 can match propagation delays to the target from each of emitters 116 arranged, for example, in a phased array. The directional transmission and focus process is controlled through a technique similar to phase reconstruction for imaging techniques, but with the specific aim of controlling the power of delivered energy to the target through complex media, such as human tissue, without homogeneous propagation properties.

In some implementations, controller 112 can communicate with a remote server to receive new control signals. For example, controller 112 can transmit feedback from sensors 114 to the remote server, and the remote server can receive the feedback, process the data, and generate updated control signals for the system 110 and other components.

System 110 can receive input from subject 102 and automatically determine a target area and control emitters 116 to produce fields of particular voltage and frequency at the target area. For example, controller 112 can determine, based on collected feedback information from subject's brain 104 in response to stimulation, a area, or large-scale brain network, to target.

System 110 performs activity detection to uniquely tailor stimulation for a particular subject 102. In some implementations, the system 110 can start with a baseline map of brain conductivity and functionality and dynamically adjust stimulation to the target area of subject's brain 104 based on activity feedback detected by sensors 114. In some implementations, system 110 can perform tomography on subject's brain 104 to generate maps, such as maps of large-scale brain activity or electrical properties of the head or brain. For example, the system 110 can produce large-scale brain network maps for subject's brain 104 based on current absorption data measured by sensors 114 that indicate the amount of activity of a particular area of subject's brain 104 in response to a particular stimulus. In some implementations, system 110 can start with provisionally tailored maps that are generally applicable to a subset of subjects 102 having a set of characteristics in common and dynamically adjust stimulation to the target area of subject's brain 104 based on activity feedback detected by sensors 114.

System 110 is generally used for non-clinical applications. For example, controller 112 can control emitters 116 such that the current of the electric fields generated are lower than the current used in therapeutic applications. In some implementations, controller 112 can be used to produce electric field regions that affect the network state that a subject is in. For example, controller 112 can be used to produce interfering regions that induce a focused state, a relaxed state, or a meditation state, among other states, of subject's brain 104. In some implementations, controller 112 can be used to manipulate the state of subject's brain 104 to increase focus and/or creativity and aid in relaxation, among other network states.

System 110 includes safety functions that allow a subject to use the system 112 without the supervision of a medical professional. In some implementations, system 110 can be used by a subject for non-clinical applications in settings other than under the supervision of a medical professional. For example, system 110 can implement limits on the amount of time that the system 110 can be used, monitor the cumulative dose delivered to various brain areas, enforce a maximum amount of current that can be output by emitters 116, or administer integrated dose control.

In some implementations, system 110 cannot be activated by a subject without the supervision of a medical professional, or cannot be activated by a subject at all. For example, system 110 may require credentials from a medical professional prior to use. In some implementations, only subject 102's doctor can turn on system 110 remotely or at their office.

In some implementations, system 110 can uniquely identify a subject 102, and may only be used by the subject 102. For example, system 110 can be locked to particular subjects and may not be turned on or activated by any other users.

System 110 can limit the range of frequencies and intensities of the stimulation applied through emitters 116 to prevent delivery of harmful patterns of stimulation. For example, system 110 can detect and classify stimulation patterns as seizure-inducing, and prevent delivery of seizure inducing stimulus. In some implementations, system 110 can detect activity patterns in early stages of the activity and preventatively take action. For example, system 110 can detect activity patterns in an early stage of anxiety and preventatively take action to prevent subject's brain 104 from progressing into later stages of anxiety. System 110 can also detect seizure activity patterns using the extra cranial activity and biometric data collected by sensors 114, and adjust the stimulation provided by emitters 116 to prevent subject 102 from having a seizure.

In some implementations, system 110 is used for therapeutic purposes. For example, system 110 can be tailored to a subject 102 and used as a brain activity regulation device that detects epileptic activity within the subject's brain 104 and provides prophylactic stimulation.

Controller 112 can use statistical and/or machine learning models which accept sensor data collected by sensors 114 and/or other sensors as inputs. The machine learning models may use any of a variety of models such as decision trees, linear regression models, logistic regression models, neural networks, classifiers, support vector machines, inductive logic programming, ensembles of models (e.g., using techniques such as bagging, boosting, random forests, etc.), genetic algorithms, Bayesian networks, etc., and can be trained using a variety of approaches, such as deep learning, association rules, inductive logic, clustering, maximum entropy classification, learning classification, etc. In some examples, the machine learning models may use supervised learning. In some examples, the machine learning models use unsupervised learning.

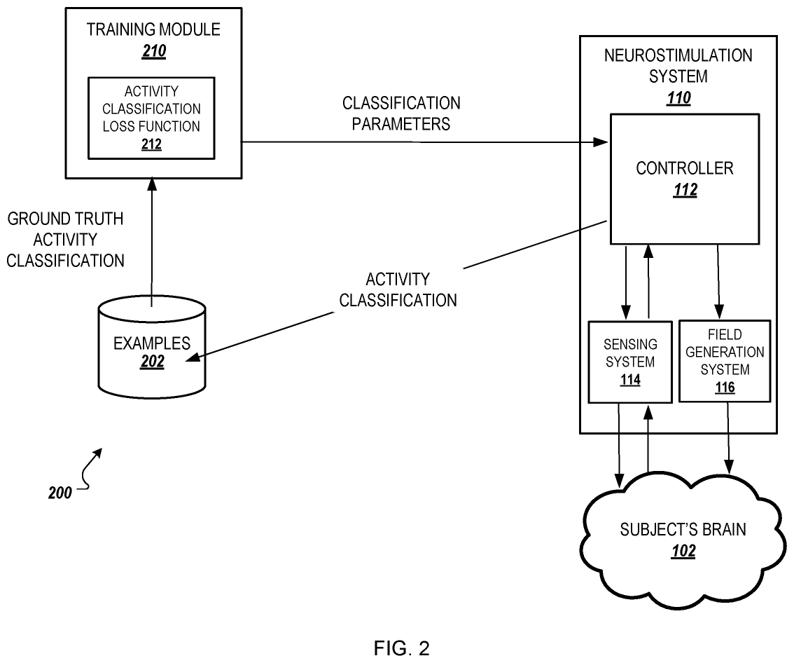

FIG. 2 is a diagram of an example block diagram of a system 200 for training a neurostimulation system. For example, system 200 can be used to train neurostimulation system 110 as described with respect to FIG. 1.

As described above with respect to FIG. 1, system 110 includes a controller 112 that classifies activity detected by a sensing system and determines stimulation parameters for a field generation system. For example, controller 112 classifies activity detected by sensors 114 and determines stimulation parameters for emitters 116. Activity classification can include identifying the location, amplitude, frequency, and phase of large-scale brain activity.

Examples 202 are provided to training module 210 as input to train an activity classification model. Examples 202 can be positive examples (i.e., examples of correctly determined activity classifications) or negative examples (i.e., examples of incorrectly determined activity classifications).

Examples 202 include the ground truth activity classification, or an activity classification defined as the correct classification. Examples 202 include sensor information such as baseline activity patterns for a particular subject. For example, examples 202 can include tomography data of subject's brain 104 generated through activity detection performed by sensors 114 or sensors external to system 110 as described above (e.g., MRIs, EEGs, MEGs, and computed tomography based on the detected data from sensors 114, among other detection techniques).

The ground truth indicates the actual, correct classification of the activity. For example, a ground truth activity classification can be generated and provided to training module 210 as an example 202 by detecting an activity, classifying the activity, and confirming that the activity classification is correct. In some implementations, a human can manually verify the activity classification. The activity classification can be automatically detected and labelled by pulling data from a data storage medium that contains verified activity classifications.

The ground truth activity classification can be correlated with particular inputs of examples 202 such that the inputs are labelled with the ground truth activity classification. With ground truth labels, training module 210 can use examples 202 and the labels to verify model outputs of an activity classifier and continue to train the classifier to improve forward modelling of brain activity through the use of detection data from sensors 114 to predict brain functionality and activity in response to stimulation input.

The sensor information guides the training module 210 to train the classifier to create a morphology correlated map. The training module 210 can associate the morphology of a particular subject's brain 104 with an activity classification to map out brain conductivity and functionality. Inverse modelling of brain activity can be conducted by using measured responses to approximate brain networks that could produce the measured responses. The training module 210 can train the classifier to learn how to map multiple raw sensor inputs to their location within subject's brain 104 (e.g., a location relative to a reference point within subject's brain 104's specific morphology) and activity classification based on a morphology correlated map. Thus, the classifier would not need additional prior knowledge during the testing phase because the classifier is able to map sensor inputs to respective areas within subject's brain 104 and classify activities using the correlated map.

Training module 210 trains an activity classifier to perform activity classification. For example, training module 110 can train controller 112 to recognize large-scale brain activity based on inputs from sensors within a area of subject's brain 104. Training module 210 refines controller 112's activity classification model using electrical tomography data collected by sensors 114 for a particular subject's brain 104. Training module 210 allows controller 112 to output complex results, such as a detected brain functionality instead of, or in addition to, simple imaging results.

Training module 210 trains controller 112 using an activity classification loss function 212. Training module 110 uses activity classification loss function 212 to train controller to classify a particular large-scale brain activity. Activity classification loss function 212 can account for variables such as a predicted location, a predicted amplitude, a predicted frequency, and/or a predicted phase of a detected activity.

Training module 210 can train controller 112 manually or the process could be automated. For example, if an existing tomographic representation of subject's brain 104 is available, the system can receive sensor data indicating brain activity in response to a known stimulation pattern to identify the ground truth area within subject's brain 104 at which an activity occurs through automated techniques such as image recognition or identifying tagged locations within the representation. A human can also manually verify the identified areas.

Training module 210 uses the loss function 112 and examples 202 labelled with the ground truth activity classification to train controller 112 to learn where and what is important for the model. Training module 210 allows controller 112 to learn by changing the weights applied to different variables to emphasize or deemphasize the importance of the variable within the model. By changing the weights applied to variables within the model, training module 210 allows the model to learn which types of information (e.g., which sensor inputs, what locations, etc.) should be more heavily weighted to produce a more accurate activity classifier.

Training module 210 uses machine learning techniques to train controller 112, and can include, for example, a neural network that utilizes activity classification loss function 212 to produce parameters used in the activity classifier model. These parameters can be classification parameters that define particular values of a model used by controller 112.

Controller 112 classifies brain activity based on data collected by sensors 114. Controller 112 performs forward modelling of brain activity and inverse modelling of brain activity, given base, reasonable assumptions regarding the stimulation applied to a target area within subject's brain 104.

Forward modelling allows controller 112 to determine how to propagate waves through subject's brain 104. For example, controller 112 can receive a specified objective (e.g., a network state of subject's brain 104) and design stimulation field patterns to modify brain activity detected by sensors 114. Controller 112 can then control two or more emitters 116 to apply electrical fields to a target area of subject's brain 104 to produce the specified objective network state.

Inverse modelling allows controller 112 to estimate the most likely relationship between the detected activity corresponds with areas or networks of subject's brain 104. For example, controller 112 can receive brain activity data from sensors 114 and reconstruct, using an activity classifier model, the location, amplitude, frequency, and phase of the large-scale brain activity. Controller 112 can then dynamically alter the existing activity classifier model and/or tomography representation of subject's brain 104 based on the reconstruction.

Controller 112 can use various types of models, including general models that can be used for all patients and customized models that can be used for particular subsets of patients sharing a set of characteristics, and can dynamically adjust the models based on the morphology of a particular subject's brain 104 or based on detected brain activity. For example, the classifier can use a base network for subjects and then tailor the model to each subject. The brain activity can be detected by sensors 114 contemporaneously or near-contemporaneously with the stimulation provided by emitters 116. In some implementations, the brain activity can be detected through techniques performed by systems external to system 110, such as functional magnetic resonance imaging (fMRI) or diffusion tensor imaging (DTI).

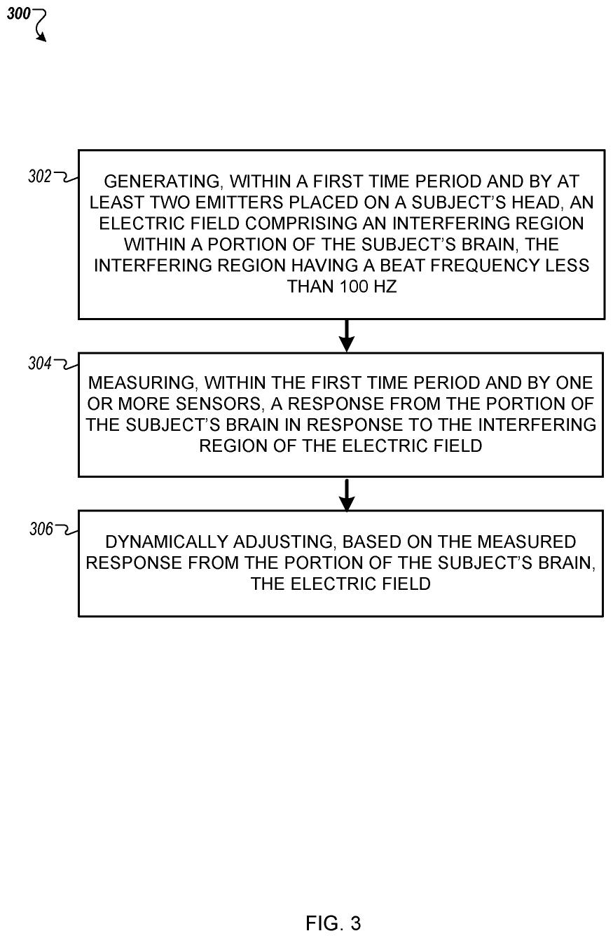

FIG. 3 is a flow chart of an example process 300 of closed loop neurostimulation of large-scale brain networks. Process 400 can be implemented by neurostimulation systems such as system 110 as described above with respect to FIGS. 1 and 2. In this particular example, process 300 is described with respect to system 110 in the form of a portable headset that can be used by a subject without the supervision of a medical professional. Briefly, according to an example, the process 300 begins with generating, within a first time period and by at least two emitters placed on a subject's head, an electric field comprising an interfering region within a portion of the subject's brain, the interfering region having a beat frequency less than 100 Hz. For example, controller 112 can operate two emitters, 116b and 116f, to generate an electric field having an interfering region within a period of 3 seconds, and at a target area within the subject's brain 104. In some implementations, the interfering region can have a beat frequency of 85 Hz.

The process 300 continues with measuring, within the first time period and by one or more sensors, a response from the portion of the subject's brain in response to the interfering region of the electric field. For example, controller 112 can operate sensors 114 to measure, within a few seconds, and thus contemporaneously or near-contemporaneously with the generating step, brain activity from the target area within the subject's brain 104. For example, sensors 114 can detect, using EEG, brain activity from the target area within the subject's brain 104 in response to the electric field having the interfering region.

The process 300 concludes with dynamically adjusting, based on the measured response form the portion of the subject's brain, the electric field. For example, controller 112 can determine, based on the measured brain activity detected by sensors 114, that subject 102 is entering a focused network state. Controller 112 can then determine, using the measured brain activity and tomography of the subject's brain 104, stimulation parameters for emitters 116 to continue inducing the focused network state in the subject's brain 104. Controller 112 can operate emitters 116 according to the determined stimulation parameters to adjust the electric field. For example, controller 112 can operate emitters 116 to alter the beat frequency and amplitude of the interfering region of the electric field, thus facilitating a closed loop neurostimulation system for large-scale brain networks. Controller 112 can operate emitters 116 with a phase shift relative to a detected in-phase large-scale brain network, enhancing or decreasing the phase lock of the large-scale brain network. Controller 112 can operate emitters 116 with a frequency shift relative to a detected in-phase large-scale brain network, increasing or decreasing the frequency of the phase-locked the large-scale brain network.

A number of implementations have been described. Nevertheless, it will be understood that various modifications may be made without departing from the spirit and scope of the disclosure. For example, various forms of the flows shown above may be used, with steps re-ordered, added, or removed.

All of the functional operations described in this specification may be implemented in digital electronic circuitry, or in computer software, firmware, or hardware, including the structures disclosed in this specification and their structural equivalents, or in combinations of one or more of them. The techniques disclosed may be implemented as one or more computer program products, i.e., one or more modules of computer program instructions encoded on a computer-readable medium for execution by, or to control the operation of, data processing apparatus. The computer readable-medium may be a machine-readable storage device, a machine-readable storage substrate, a memory device, a composition of matter affecting a machine-readable propagated signal, or a combination of one or more of them. The computer-readable medium may be a non-transitory computer-readable medium. The term “data processing apparatus” encompasses all apparatus, devices, and machines for processing data, including by way of example a programmable processor, a computer, or multiple processors or computers. The apparatus may include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

A computer program (also known as a program, software, software application, script, or code) may be written in any form of programming language, including compiled or interpreted languages, and it may be deployed in any form, including as a standalone program or as a module, component, subroutine, or other unit suitable for use in a computing environment. A computer program does not necessarily correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one computer or on multiple computers that are located at one site or distributed across multiple sites and interconnected by a communication network.

The processes and logic flows described in this specification may be performed by one or more programmable processors executing one or more computer programs to perform functions by operating on input data and generating output. The processes and logic flows may also be performed by, and apparatus may also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer may be embedded in another device, e.g., a tablet computer, a mobile telephone, a personal digital assistant (PDA), a mobile audio player, a Global Positioning System (GPS) receiver, to name just a few. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory may be supplemented by, or incorporated in, special purpose logic circuitry.

To provide for interaction with a user, the techniques disclosed may be implemented on a computer having a display device, e.g., a CRT (cathode ray tube) or LCD (liquid crystal display) monitor, for displaying information to the user and a keyboard and a pointing device, e.g., a mouse or a trackball, by which the user may provide input to the computer. Other kinds of devices may be used to provide for interaction with a user as well; for example, feedback provided to the user may be any form of sensory feedback, e.g., visual feedback, auditory feedback, or tactile feedback; and input from the user may be received in any form, including acoustic, speech, or tactile input.

Implementations may include a computing system that includes a back end component, e.g., as a data server, or that includes a middleware component, e.g., an application server, or that includes a front end component, e.g., a client computer having a graphical user interface or a Web browser through which a user may interact with an implementation of the techniques disclosed, or any combination of one or more such back end, middleware, or front end components. The components of the system may be interconnected by any form or medium of digital data communication, e.g., a communication network. Examples of communication networks include a local area network (“LAN”) and a wide area network (“WAN”), e.g., the Internet.

The computing system may include clients and servers. A client and server are generally remote from each other and typically interact through a communication network. The relationship of client and server arises by virtue of computer programs running on the respective computers and having a client-server relationship to each other.

While this specification contains many specifics, these should not be construed as limitations, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products.

Thus, particular implementations have been described. Other implementations are within the scope of the following claims. For example, the actions recited in the claims may be performed in a different order and still achieve desirable results.

What is claimed is:

1. A neurostimulation headset, comprising:
at least two ultrasound transducer modules configured to generate, within a first time period, a first focused ultrasound wave at a region within a portion of a subject's brain;
one or more sensors configured to measure, within the first time period, a response from the portion of the subject's brain in response to the first focused ultrasound wave; and
an electronic controller in communication with the at least two emitters and the one or more sensors configured to dynamically adjust, based on the measured response from the portion of the subject's brain, a power level of one or more of the at least two ultrasound transducer modules to generate a second focused ultrasound wave at the region within the portion of the subject's brain.

2. The neurostimulation headset of claim 1, wherein the one or more sensors includes an imaging array that generates imaging ultrasound waves at a first power level, the first power level being sufficient to produce an imaging effect of the region within the portion of the subject's brain.

3. The neurostimulation headset of claim 1, wherein the second focused ultrasound wave is at a second power level sufficient to provide a therapeutic effect for the subject.

4. The neurostimulation headset of claim 1, wherein the electronic controller is further configured to:
calculate, based on the measured response from the portion of the subject's brain in response to the first focused ultrasound wave, an adjusted focused ultrasound wave; and
dynamically adjust, based on the measured response from the portion of the subject's brain and the adjusted focused ultrasound wave, the first focused ultrasound wave to generate the second focused ultrasound wave.

5. The neurostimulation headset of claim 4, wherein dynamically adjusting the first focused ultrasound wave to generate the second focused ultrasound wave comprises determining, based on the location of the region and a machine learning model, at least one focusing parameter for the one or more ultrasound transducer modules.

6. The neurostimulation headset of claim 4, wherein dynamically adjusting the focused ultrasound wave to generate the second focused ultrasound wave comprises determining, based on the location of the region and feedback input by the subject, at least one focusing parameter for the one or more ultrasound transducer modules.

7. The neurostimulation headset of claim 4, wherein dynamically adjusting the focused ultrasound wave to generate the second focused ultrasound wave comprises determining, based on the location of the region and one or more physiological measurements of the subject, at least one focusing parameter for the one or more ultrasound transducer modules.

8. A method, comprising:
generating, within a first time period and by at least two ultrasound transducer modules, a first focused ultrasound wave at a region within a portion of a subject's brain;
measuring, within the first time period and by one or more sensors, a response from the portion of the subject's brain in response to the first focused ultrasound wave; and
dynamically adjusting, based on the measured response from the portion of the subject's brain and by an electronic controller in communication with the at least two ultrasound transducer modules and the one or more sensors, a power level of one or more of the at least two ultrasound transducer modules to generate a second focused ultrasound wave at the region within the portion of the subject's brain.

9. The method of claim 8, wherein the one or more sensors includes an imaging array that generates imaging ultrasound waves at a first power level, the first power level being sufficient to produce an imaging effect of the region within the portion of the subject's brain.

10. The method of claim 8, wherein the second focused ultrasound wave is at a second power level sufficient to provide a therapeutic effect for the subject.

11. The method of claim 8, further comprising calculating, based on the measured response from the portion of the subject's brain in response to the first focused ultrasound wave, an adjusted focused ultrasound wave; and
dynamically adjusting, based on the measured response from the portion of the subject's brain and the adjusted focused ultrasound wave, the first focused ultrasound wave to generate the second focused ultrasound wave.

12. The method of claim 11, wherein dynamically adjusting the first focused ultrasound wave to generate the second focused ultrasound wave comprises determining, based on the location of the region and a machine learning model, at least one focusing parameter for the one or more ultrasound transducer modules.

13. The method of claim 11, wherein dynamically adjusting the focused ultrasound wave to generate the second focused ultrasound wave comprises determining, based on the location of the region and feedback input by the subject, at least one focusing parameter for the one or more ultrasound transducer modules.

14. The method of claim 11, wherein dynamically adjusting the focused ultrasound wave to generate the second focused ultrasound wave comprises determining, based on the location of the region and one or more physiological measurements of the subject, at least one focusing parameter for the one or more ultrasound transducer modules.

15. A computer-readable storage device storing instructions that when executed by one or more processors cause the one or more processors to perform operations comprising:
generating, within a first time period and by at least two ultrasound transducer modules, a first focused ultrasound wave at a region within a portion of a subject's brain;
measuring, within the first time period and by one or more sensors, a response from the portion of the subject's brain in response to the first focused ultrasound wave; and
dynamically adjusting, based on the measured response from the portion of the subject's brain and by an electronic controller in communication with the at least two ultrasound transducer modules and the one or more sensors, a power level of one or more of the at least two ultrasound transducer modules to generate a second focused ultrasound wave at the region within the portion of the subject's brain.

16. The computer-readable storage device of claim 15, wherein the one or more sensors includes an imaging array that generates imaging ultrasound waves at a first power level, the first power level being sufficient to produce an imaging effect of the region within the portion of the subject's brain.

17. The computer-readable storage device of claim 15, wherein the second focused ultrasound wave is at a second power level sufficient to provide a therapeutic effect for the subject.

18. The computer-readable storage device of claim 15, the instructions further comprising: calculating, based on the measured response from the portion of the subject's brain in response to the first focused ultrasound wave, an adjusted focused ultrasound wave; and
dynamically adjusting, based on the measured response from the portion of the subject's brain and the adjusted focused ultrasound wave, the first focused ultrasound wave to generate the second focused ultrasound wave.

19. The computer-readable storage device of claim 18, wherein dynamically adjusting the first focused ultrasound wave to generate the second focused ultrasound wave comprises determining, based on the location of the region and a machine learning model, at least one focusing parameter for the one or more ultrasound transducer modules.

20. The computer-readable storage device of claim 18, wherein dynamically adjusting the focused ultrasound wave to generate the second focused ultrasound wave comprises determining, based on the location of the region and feedback input by the subject, at least one focusing parameter for the one or more ultrasound transducer modules.
